(19) United States
(12) Patent Application Publication
(10) Pub. No.: US 2004/0242990 A1
(43) Pub. Date: Dec. 2, 2004

(54) Device, system, and method for detecting vulnerable plaque in a blood vessel

(75) Inventors: Mark Brister, Encinitas, CA (US); Patrice Tremble, Santa Rosa, CA (US)

Correspondence Address: MEDTRONIC VASCULAR, INC., IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/827,987

(22) Filed: Apr. 20, 2004

Related U.S. Application Data
(60) Provisional application No. 60/464,441, filed on Apr. 22, 2003.

(51) Int. Cl. A61B 5/04

(52) U.S. Cl. 600/407; 600/381

(57) ABSTRACT

A device, system, and method for detecting vulnerable plaque in a blood vessel of a patient are disclosed. The device includes a catheter having at least one aperture, and a flexible guidewire within the aperture. The guidewire includes a coiled portion with at least one sensor for receiving information about the blood vessel and for determining presence of vulnerable plaque. The system includes a catheter and a flexible guidewire having a coiled configuration for positioning at least one sensor adjacent a blood vessel wall to allow the sensor to receive information for determining the presence of vulnerable plaque. The method includes steps for inserting a flexible guidewire into a lumen of the blood vessel; positioning a coiled guidewire with at least one sensor adjacent a blood vessel wall; receiving information about the blood vessel from the sensor and determining the presence of a vulnerable plaque based on the received information.

### RELATED APPLICATION

This application claims priority to U.S. Provisional Application No. 60/464,441, “Device, System and Method for Detecting Vulnerable Plaque in a Blood Vessel” to Mark Brister and Patrice Tremble, filed Apr. 22, 2003, the entirety of which is incorporated by reference.

### TECHNICAL FIELD OF THE INVENTION

The present invention relates generally to the field of intravascular devices. More particularly, the invention relates to a device, system, and method for detecting vulnerable plaque in a blood vessel.

### BACKGROUND OF THE INVENTION

Heart disease, specifically coronary artery disease, is a major cause of death, disability, and healthcare expense. Until recently, most heart disease was considered primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot. The clot may choke off the flow of oxygen rich blood (ischemia) to heart muscles, causing a heart attack. Alternatively, the clot may break off and lodge in another organ vessel such as the brain resulting in a thrombotic stroke.

Within the past decade, evidence has emerged changing the paradigm of atherosclerosis, coronary artery disease, and heart attacks. While the build up of hard plaque may produce angina and severe ischemia in the coronary arteries, new clinical data now suggests that the rupture of sometimes non-occlusive, vulnerable plaques causes the vast majority of heart attacks. The rate is estimated as high as 60-80 percent. In many instances vulnerable plaques do not impinge on the vessel lumen, rather, much like an abscess they are ingrained under the arterial wall. For this reason, conventional angiography or fluoroscopy techniques are unlikely to detect the vulnerable plaque. Due to the difficulty associated with their detection and because angina is not typically produced, vulnerable plaques may be more dangerous than other plaques that cause pain.

The majority of vulnerable plaques include a lipid pool, necrotic smooth muscle (endothelial) cells, and a dense infiltrate of macrophages contained by a thin fibrous cap (i.e., some two micrometers thick or less). The lipid pool is believed to be formed as a result of pathological process involving low density lipoprotein (LDL), macrophages and the inflammatory process. The macrophages oxidize the LDL producing foam cells. The macrophages, foam cells, and associated endothelial cells release various substances, such as tumor necrosis factor, tissue factor and matrix proteinases, which result in generalized cell necrosis and apoptosis, pro-coagulation and weakening of the fibrous cap. The inflammation process may weaken the fibrous cap to the extent that sufficient mechanical stress, such as that produced by increased blood pressure, may result in rupture. The lipid core and other contents of the vulnerable plaque may then spill into the blood stream thereby initiating a clotting cascade. The cascade produces a blood clot (thrombosis) that potentially results in a heart attack and/or stroke. The process is exacerbated due to the release of collagen and other plaque components (e.g., tissue factor), which enhance clotting upon their release.

Several strategies have been developed for the diagnosis and localization of vulnerable plaques. One strategy involves the measurement of temperature within a blood vessel. A localized increase in temperature is generally associated with the vulnerable plaque because of the tissue damage and inflammation. It has been observed that the inflamed necrotic core of the vulnerable plaque maintains a temperature of one or more degrees Celsius higher than that of the surrounding tissue. For example, a relatively normal vessel temperature may be about 37° C. whereas the vulnerable plaque may have a localized temperature as high as 40° C. Measurement of these temperature differences within the blood vessel may provide means for detecting vulnerable plaque.

Other strategies for diagnosis and localization include labeling vulnerable plaque with a marker. The marker substance may be specific for a component and/or characteristic of the vulnerable plaque. One strategy includes using markers that have an affinity for the vulnerable plaque, more so than for healthy tissue. The affinity markers include molecules such as antibodies and other binding compounds, which may have an affinity for matrix proteinases, foam cells, macrophages, collagen, the fibrous cap, or other plaque components. Another detection strategy includes using markers that changes properties while associated with the vulnerable plaque, but does not necessarily associate with the vulnerable plaque. For example, such a marker may change properties upon being exposed to a specific temperature, pH, other molecule, or other condition.

The markers may signal the presence of the vulnerable plaque by emitting electromagnetic radiation. For example, a patient may be given an antibody marker with a specific affinity for foam cells. The antibody may be labeled with a radioisotope or with a fluorescent moiety. The electromagnetic radiation information emitted by the antibody may then be detected thereby facilitation diagnosis and localization of the vulnerable plaque.

One problem associated with the diagnosis and localization of the vulnerable plaque pertains to detector size. Detection is typically achieved with a catheter or guidewire device carrying one or more sensors. The catheters are relatively large in diameter compared to a guidewire thereby making navigation of tortuous vessels during diagnosis difficult. Moreover, catheter sensor(s) may not provide means for varying their outer diameter. As such, the catheter sensor(s) may not be positioned in proximity to the vessel wall. Sensor guidewires are generally much smaller and are, thus, easier to navigate. Therefore, it would be desirable to provide a strategy for detecting vulnerable plaque using a minimally sized detection device, such as a guidewire. Furthermore, it would be desirable to provide such a strategy that may provide proximal positioning of the sensor(s) to the vessel wall.

Another problem associated with the diagnosis and localization of the vulnerable plaque pertains to vessel trauma. During the diagnosis and localization of the vulnerable plaque, the detection catheters and guidewires may be “dragged” along the blood vessel. In this manner, the vessel in scanned longitudinally for changes in temperature and/or presence of a marker. The friction produced between the catheter/guidewire and vessel surfaces is generally undesirable for the health of the blood vessel. Furthermore, current guidewires are typically shaped with bends or sharp points in order to position the sensor(s) proximate the vessel wall. The surface friction and force between a bent sensor guidewire and vessel wall may be sufficient to compromise the integrity of a vulnerable plaque fibrous cap. The plaque may rupture and thereby pose risk to the patient. Therefore, it would be desirable to provide a strategy for detecting vulnerable plaque while minimizing any trauma to the blood vessel.

Another problem associated with the diagnosis and localization of the vulnerable plaque pertains to blood coagulation. As a detection device is in contact with the blood over a period of time, coagulation may occur. In the case of a catheter or guidewire, the coagulation may give rise to imprecise measurements. Therefore, it would be desirable to provide a strategy for detecting vulnerable plaque while minimizing any coagulation associated with the detection device.

Accordingly, it would be desirable to provide a strategy for detecting vulnerable plaque that would overcome the aforementioned and other disadvantages.

### SUMMARY OF THE INVENTION

One aspect according to the invention provides a device for detecting a vulnerable plaque associated with a blood vessel of a patient. The device includes a catheter including at least one aperture formed therein. A flexible guidewire is slidably carried within the aperture. The guidewire includes a coiled configuration portion. At least one sensor is disposed on the coiled configuration portion of the guidewire. The sensor is adapted for receiving information about the blood vessel for determining presence of the vulnerable plaque. The information may include electromagnetic radiation information such as radio wave radiation, microwave radiation, infrared radiation, visible light radiation, ultraviolet radiation, x-ray radiation, alpha radiation, beta radiation, gamma radiation, and fluorescence radiation. The sensor may be adapted to provide a low-profile shape. The guidewire may include a surface material for reducing friction between the guidewire and the blood vessel and/or coagulation associated with the guidewire. The surface material may include Teflon®), Carmeda®, Hepamed®, a ceramic material, a hydrophilic material, a lubricating material, a carbon-based material, and a silicon-based material. The coiled configuration portion may include a corkscrew configuration wherein the guidewire is coiled a plurality of turns around a central axis. The coiled configuration portion may be adapted to expand radially toward a blood vessel wall. A carrier may be coupled to the sensor for transferring the information from a diagnostic site to a site external the patient. The device may include an input device for receiving input and a processing unit in communication with the sensor and the input device. The processing unit determines presence of vulnerable plaque based on at least one of the information and the input. A controller is in communication with the processing unit. The controller positions the guidewire within the patient based on at least one of the information and the input. An output device in communication with the processing unit transmits at least one of the information, the input, and the determined presence of vulnerable plaque. The device may further include a drug delivery element adapted for delivering therapeutic agents to the blood vessel.

Another aspect according to the invention includes a system for detecting a vulnerable plaque associated with a blood vessel of a patient. The system includes a catheter and a flexible guidewire slidably carried by the catheter. The guidewire includes a coiled configuration means for positioning at least one sensor. The coiled configuration means are positioned adjacent a wall of the blood vessel to allow the sensor to receive information for determining presence of the vulnerable plaque.

Another aspect according to the invention includes a method for detecting a vulnerable plaque associated with a blood vessel of a patient. The method includes the steps inserting a flexible guidewire into a lumen of the blood vessel. A coiled configuration portion of the guidewire is positioned adjacent a wall of the blood vessel. Information about the blood vessel wall is received from at least one sensor disposed on the coiled configuration portion. Presence of the vulnerable plaque is determined based on the received information. The information may include electromagnetic radiation information such as radio wave radiation, microwave radiation, infrared radiation, visible light information, ultraviolet radiation, x-ray radiation, alpha radiation, beta radiation, gamma radiation, and fluorescence radiation. Positioning the coiled configuration portion may include sliding the guidewire longitudinally with respect to the blood vessel and/or rotating the guidewire about a central axis. The coiled configuration portion may include a corkscrew configuration wherein the guidewire is coiled a plurality of turns around a central axis. The coiled configuration portion may be adapted to expand radially toward a blood vessel wall. Determining presence of vulnerable plaque may include comparing received information from a healthy blood vessel site to received information from a vulnerable plaque site. The received information may be transmitted from a diagnostic site to a site external the patient. A therapeutic agent may be delivered to the blood vessel based on the information.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of a device for detecting a vulnerable plaque associated with a blood vessel of a patient, in accordance with one embodiment of the present invention;

FIG. 2 is a cross-section view of a guide catheter and guidewire along the lines 2-2 shown in FIG. 1;

FIG. 3 is a perspective view of a guidewire coiled configuration portion, in accordance with one embodiment of the present invention;

FIG. 4 is a detailed perspective view of the guidewire coiled configuration portion shown in FIG. 3, the guidewire including a plurality of sensors;

FIG. 5 is a cross-section view of the guidewire coiled configuration portion along the lines 5-5 shown in FIG. 4;

FIG. 6 is a schematic view of a vulnerable plaque detection procedure within a patient, in accordance with the present invention; and

FIGS. 7A and 7B are detailed schematic views of vulnerable plaque detection within a blood vessel, in accordance with the present invention.

### DESCRIPTION OF THE PRESENTLY PREFERRED EMBODIMENTS

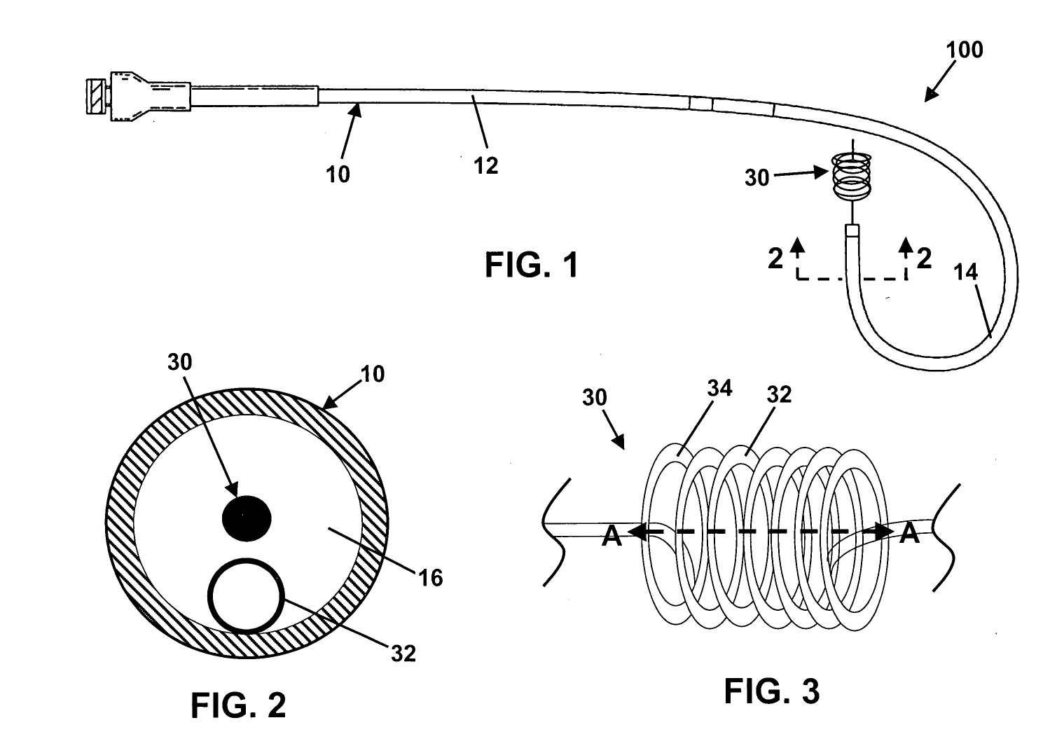

Referring to the drawings, wherein like reference numerals refer to like elements, FIG. 1 is a perspective view of a device 100 for detecting a vulnerable plaque associated with a blood vessel of a patient, in accordance with one embodiment of the present invention. The device 100 includes a guide catheter 10 and associated flexible guidewire 30. Those skilled in the art will recognize that numerous catheters and guidewires are compatible with the disclosed invention and that the illustrated catheter 10 and guidewire 30 are an example of merely one such device. Numerous additions, substitutions, changes, and modifications may be made to the device 100 while still providing a catheter 10 and guidewire 30 in accordance with the present invention.

Catheter 10 is an example of a guide catheter disclosed by U.S. Pat. No. 6,106,510 issued to Lunn et al., which is incorporated by reference herein. Other catheter design examples that may be adapted for use with the present invention include, but are not limited to, U.S. Pat. No. 4,976,689 issued to Buchbinder et al., U.S. Pat. No. 5,061,273 issued to Yock, U.S. Pat. No. 5,964,971 issued to Lunn, U.S. Pat. No. 6,251,084 issued to Coelho, and U.S. Pat. No. 6,394,976 issue to Winston et al., which are incorporated by reference herein. Numerous known guidewire designs, materials, and sizes are known and may be adapted for use with the present invention. Guidewire 30 is an example of a modified guidewire design.

In one embodiment, catheter 10 may include a stiff proximal segment 12 to allow pushing, pulling, and turning while having a pliable, more flexible distal segment 14 for navigating tortuous blood vessels of a patient. The flexible nature of the distal segment 14 may reduce any trauma imparted on a blood vessel wall. Numerous materials and strategies for manufacturing catheter 10 are known. Examples of suitable catheter 10 materials include, but are not limited to thermoplastic elastomers and/or urethanes, polymer, polypropylene, plastic, ethelene chlorotrifluoroethylene (ECTFE), polytetrafluoroethylene (PTFE), fluorinated ethylene propylene copolymer (FEP), PEBAX®, Vestamid®, Nylon-6, Nylon-12, Tecoflex®, Halar®, Hyflon®, and combinations thereof.

FIG. 2 is a cross-section view of the catheter 10 and guidewire 30 along the lines 2-2 shown in FIG. 1. Catheter 10 includes an aperture 16 formed therein for slidably carrying the guidewire 30. Catheter 10 may further include a drug delivery element for delivering therapeutic agents to the blood vessel. In one embodiment, the drug delivery element may include at least one elongated tube 18 positioned within the catheter 10. As such, the therapeutic agent(s) may be administered from outside the patient to an appropriate delivery site within the blood vessel. The therapeutic agents may or may not be used to facilitate detection and/or treatment of vulnerable plaque. Examples of therapeutic agents that may be delivered to the blood vessel include, but are not limited to, antiangiogenesis agents, antiarteriosclerotic agents, antiarythmic agents, antibiotics, antibodies, antidiabetic agents, antiendothelin agents, antinflammatory agents, antimitogenic factors, antioxidants, antiplatelet agents, antiproliferative agents, antisense agents, binding agents, calcium channel blockers, clot dissolving enzymes, growth factor inhibitors, growth factors, immunosuppressants, markers, nitrates, nitric oxide releasing agents, vasodilators, virus-mediated gene transfer agents, agents having a desirable therapeutic application, combinations of the above, and a variety of other agents or drugs may also be included to provide other benefits.

FIG. 3 is a perspective view of a guidewire coiled configuration portion 32. In one embodiment, the coiled configuration 32 is positioned at a distal segment of the guidewire 30. In another embodiment, the coiled configuration 32 is positioned at a relative distance from the distal segment of the guidewire 30. Those skilled in the art will recognize that the position of the coiled configuration on the guidewire may vary.

In one embodiment, as shown, the coiled configuration 32 includes a “corkscrew” configuration wherein the guidewire 30 is coiled a plurality of turns around a central axis A-A. Such a coiled configuration 32 provides a “coiled tube” whereby the outer edges of the tube may contact the inner wall of the blood vessel. As described below, this contact may enhance detection of vulnerable plaque. The coiled configuration 32 may also provide a spring-like expansive force whereby the tube expands radially toward the vessel wall. Should the diameter of the vessel wall narrow, the coiled tube may compress while still maintaining contact with the vessel wall. In addition, the relatively easy compression of the coil may minimize any friction force and, therefore, trauma on the vessel wall. As such, the coiled configuration 32 and, specifically, the corkscrew configuration provides a geometry that maintains contact with the vessel wall while minimizing the force of contact. Unlike some current catheters designs, the coiled configuration 32 tube diameter is variable to maintain contact with the vessel wall.

The geometry of the coiled configuration 32 is such that, unlike some current guidewire sensor designs, a lack of guidewire 30 sharp bends or angles is provided thereby minimizing any potential injury to the blood vessel or risk or rupturing the vulnerable plaque. Those skilled in the art will recognize that the guidewire coiled configuration 32 may vary from the illustrated and described configuration. Numerous geometric shapes including a variety of coil shapes may be provided to provide the advantages of the present invention. In another embodiment, a variety of guidewire twists, turns, and soft bends may be used to comprise the coiled configuration of the present invention.

The guidewire 30 may include a surface material 34 to reduce friction and/or coagulation associated with the guidewire 30. In one embodiment, the guidewire 30 may be coated with the surface material 34. In another embodiment, the surface material may be integral to material of the guidewire 30. In another embodiment, only the coiled configuration 32 may include the surface material. Suitable examples of surface materials include, but are not limited to, Teflon®, Carmeda®, Hepamed®), a ceramic material, a hydrophilic material, a lubricating material, a carbon-based-material, a silicone-based material, and combinations thereof. Those skilled in the art will recognize that numerous surface materials may be used with the present invention for reducing surface friction and/or coagulation.

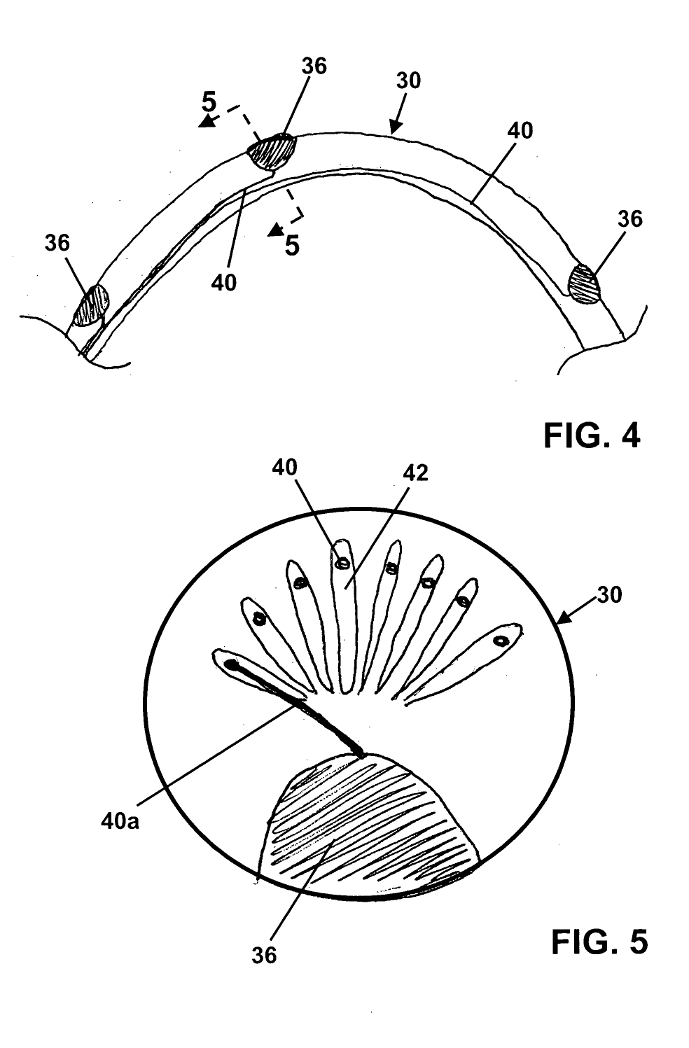

FIG. 4 is a detailed perspective view of the guidewire coiled configuration 32. The coiled configuration 32 includes at least one sensor 36, in this case three. Numerous sensors, including those for receiving various types of information, are known in the art and may be adapted for use with the present invention. In one embodiment, sensors 36 are positioned on the previously described “coiled tube” outer edges. This outer position may provide sensor 36 proximal positioning or contact with the blood vessel wall. This close positioning may allow the sensor 36 to optimally detect information about the vessel wall. As such, this may potentially provide greater sensitivity and accuracy for detection of the vulnerable plaque.

The sensor 36 profile shape and degree of projection/recession may vary. In one embodiment, sensor 36 may be adapted to provide a low-profile shape. Numerous shapes may constitute a low profile shape. For example, as shown, the sensor 36 may have a rounded shape and project slightly from a guidewire surface 38. Alternatively, the sensor 36 may be coplanar or recessed with the guidewire surface 38. The low-profile sensor 36 shape may allow optimal position while minimizing friction and, therefore, reduce any undesirable effects associated with contact between the guidewire 30 and vessel wall.

A carrier 40 may be coupled to the sensor 36 for transferring information from a diagnostic site to a site external the patient. In one embodiment as shown, the carrier 40 may be a wire or fiber optic element that is operably attached to the sensor 36. The carrier 40 may run the length of the guidewire 30 to transfer information received by the sensor 36. In another embodiment, the carrier 40 may be a device such as a radio wave or ultrasound transmitter for transmitting the sensor 36 information external the patient.

FIG. 5 is a cross-section view of the guidewire coiled configuration portion 32 along the lines 5-5 shown in FIG. 4. Sensor 36 is operably attached to carrier 40a. At least one carrier aperture 42 may be formed within the guidewire 30 forming a passageway for carrier(s) 40. In one embodiment, an individual carrier aperture 42 may carry an individual carrier 40. This may allow for electronic isolation of the carriers 40. In another embodiment, any number or configuration of carrier aperture(s) may be formed to allow carrier 40 passageway(s). For example, a single carrier aperture may be provided for electronically isolated carriers. Those skilled in the art will recognize that numerous guidewire architectures and designs may be used for transferring the information received by the sensor and, specifically, providing passageway of the carrier(s).

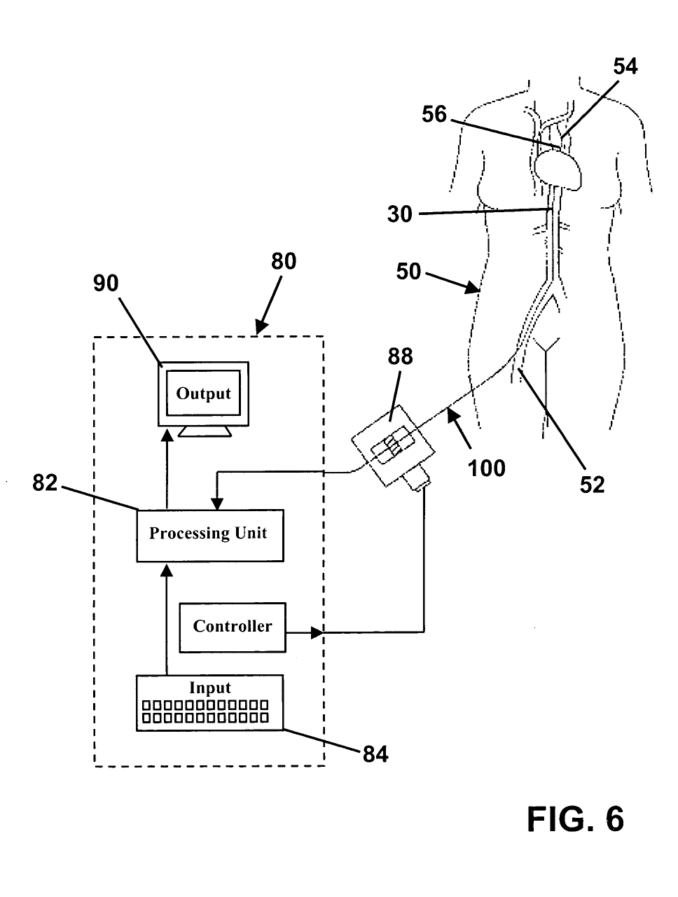

FIG. 6 is a schematic view of a vulnerable plaque detection procedure within a patient 50, in accordance with the present invention. Those skilled in the art will recognize that although the present invention is described primarily in the context of diagnosing and localizing vulnerable plaque in an artery, the inventors contemplate broader potential applicability. Any number of conditions and vascular locations including vulnerable plaque may benefit from the present invention. Furthermore, the procedure is not limited to the described detection strategy. Numerous modifications, substitutions, additions, subtractions, and variations may be made to the procedure while providing effective vulnerable plaque detection consistent with the present invention. For example, any detected vulnerable plaque may also be treated utilizing the drug delivery element aspect of the present invention. In one embodiment, the device 100 may be used during a catheterization procedure to diagnose and localize any vulnerable plaque associated with patient 50 blood vessels. In addition, the device 100 may be used to treat detected vulnerable plaque.

The procedure may begin by inserting the device 100 into a blood vessel lumen 52, such as through an incision made in patient 50 femoral artery. The device 100 may be advanced to a desired diagnostic site 54 through a vessel pathway, which in this case is the second iliac artery and abdominal aorta. It is important to note that pathways other than the one described may be used with the present invention. In addition, the described order of events may be varied during vulnerable plaque detection and treatment.

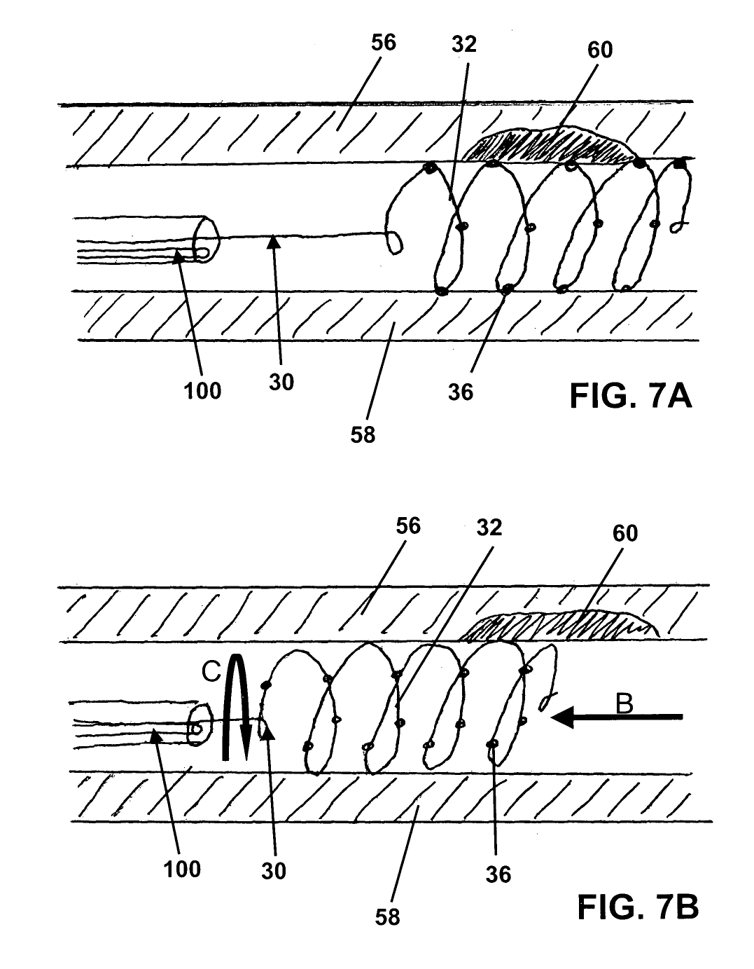

The coiled configuration of the guidewire 30 may then be positioned adjacent a blood vessel wall 56 at the desired diagnostic site 54. In one embodiment, the coiled configuration may expand radially toward the blood vessel wall 56. As shown in FIGS. 7A and 7B, coiled configuration 32 and, specifically, sensors 36 may be positioned so as to come in close proximity or to contact the blood vessel wall 56. The positioning of the device 100 and, specifically, the guidewire 30 and coiled configuration 32 may be determined by visualization methods known in the art, such as fluoroscopy and/or intravascular ultrasound (IVUS).

Referring to FIG. 7A, sensors 36 may receive information about the blood vessel wall 56. The sensors 36 may receive information from healthy vascular tissue 58 as well as the vulnerable plaque 60. The information may include electromagnetic radiation information such as radio wave radiation, microwave radiation, infrared radiation, visible light information, ultraviolet radiation, x-ray radiation, alpha radiation, beta radiation, gamma radiation, fluorescence radiation, and the like. In one embodiment, the sensors 36 may be adapted to receive infrared radiation and, therefore, measure temperature of the healthy vascular tissue 58 and vulnerable plaque 60. In another embodiment, the sensors 36 may be adapted to receive electromagnetic radiation, such as fluorescence or beta radiation, emitted from a marker associated with the vulnerable plaque 60. In another embodiment, the sensors 36 may be adapted to receive a plurality of different types of electromagnetic radiation and, thus, different types of information. Those skilled in the art will recognize that the sensors 36 may receive numerous types and combinations of information for detecting the vulnerable plaque 60.

As shown in FIG. 7B, once information has been received from a specific vessel site, the coiled configuration 32 may be positioned to a new site. In one embodiment, as shown by arrow B, the guidewire 30 may be slid longitudinally with respect to the blood vessel. In another embodiment, as shown by arrow C, the guidewire 30 may be rotated about a central axis. In another embodiment, the guidewire 30 may be rotated as it is advanced longitudinally. Rotation of the guidewire 30 may allow for a more thorough coverage of the vessel wall 56 and, thus, more reliable detection of vulnerable plaque 60.

Referring again to FIG. 6, device 100 may include an apparatus, such as a computer 80, for determining the presence of vulnerable plaque. The information received by the sensors may be transmitted to a processing unit 82. The processing unit 82 may be a computer central processing unit running a program for determining presence of vulnerable plaque. In one embodiment, sensor information received from a healthy blood vessel site may be compared to information received from another diagnostic site. Differences in the information, such as differences in vessel temperature, may indicate the presence of vulnerable plaque at the diagnostic site. In another embodiment, the received information may be compared to known values for determining the presence of vulnerable plaque. For example, temperature or marker fluorescence values exceeding a certain threshold level may indicate vulnerable plaque. Those skilled in the art will recognize that numerous strategies may be used for determining the presence of vulnerable plaque.

The computer 80 may include an input device 84, such as a keyboard and/or mouse, in communication with the processing unit 82. The input device 84 may receive input from an operator (not shown). The operator may specify parameters of the procedure through the input device 84. For example, the operator may specify device 100 position, sensor control and thresholds, and drug delivery. The input device 84 may faciliate real-time control of the procedure.

The computer 80 may include a controller 86 in communication with the processing unit 82 for positioning the guidewire 30 within the patient. The positioning may be based on the sensor information and/or the operator input. To actuate guidewire 30 positioning, the controller 86 may control a motorized pullback device 88, which may be used to advance, retreat, and or rotate the device 100 in a precise manner. The controller 86 may also control the delivery of one or more therapeutic agents to the blood vessel via the drug delivery element.

The computer 80 may include an output device 90, such as a monitor, for transmitting the sensor information, the received input, and/or the determined presence of vulnerable plaque. The operator may monitor the progress of the procedure through the output device 90.

While the embodiments of the invention disclosed herein are presently considered to be preferred, various changes and modifications may be made without departing from the spirit and scope of the invention. For example, the catheter and guidewire device and system, and method of utilizing the same are not limited to any particular design or sequence. Specifically, the coiled configuration portion, sensors, drug delivery element, surface material, computer, and procedure step order may vary without limiting the utility of the invention. Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. A device for detecting a vulnerable plaque associated with a blood vessel of a patient, the device comprising:
a catheter including at least one aperture formed therein;
a flexible guidewire slidably carried within the aperture, the guidewire including a coiled configuration portion; and
at least one sensor disposed on the coiled configuration portion of the guidewire; wherein the sensor adapted for receiving information about the blood vessel for determining presence of the vulnerable plaque.

2. The device of claim 1 wherein the information comprises electromagnetic radiation information.

3. The device of claim 1 wherein the electromagnetic radiation is selected from a group consisting of radio wave radiation, microwave radiation, infrared radiation, visible light radiation, ultraviolet radiation, x-ray radiation, alpha radiation, beta radiation, gamma radiation, and fluorescence radiation.

4. The device of claim 1 wherein the sensor is adapted to provide a low-profile shape.

5. The device of claim 1 wherein the guidewire comprises a surface material for reducing at least one of friction between the guidewire and the blood vessel and coagulation associated with the guidewire.

6. The device of claim 5 wherein the surface material is selected from a group consisting of Teflon®, Carmeda®, Hepamed®, a ceramic material, a hydrophilic material, a lubricating material, a carbon-based material, and a silicon-based material.

7. The device of claim 1 wherein the coiled configuration portion comprises a corkscrew configuration wherein the guidewire is coiled a plurality of turns around a central axis.

8. The device of claim 1 wherein the coiled configuration portion is adapted to expand radially toward a blood vessel wall.

9. The device of claim 1 further comprising a carrier coupled to the sensor for transferring the information from a diagnostic site to a site external the patient.

10. The device of claim 1 further comprising:
an input device for receiving input;
a processing unit in communication with the sensor and the input device, the processing unit determining presence of vulnerable plaque based on at least one of the information and the input;
a controller in communication with the processing unit, the controller positioning the guidewire within the patient based on at least one of the information and the input; and
an output device in communication with the processing unit, the output device for transmitting at least one of the information, the input, and the determined presence of vulnerable plaque.

11. The device of claim 1 further comprising a drug delivery element adapted for delivering therapeutic agents to the blood vessel.

12. A method for detecting a vulnerable plaque associated with a blood vessel of a patient, the method comprising:
inserting a flexible guidewire into a lumen of the blood vessel;
positioning a coiled configuration portion of the guidewire adjacent a wall of the blood vessel;
receiving information about the blood vessel wall from at least one sensor disposed on the coiled configuration portion; and
determining presence of the vulnerable plaque based on the received information.

13. The method of claim 12 wherein the information comprises electromagnetic radiation information.

14. The method of claim 13 wherein the electromagnetic radiation is selected from a group consisting of radio wave radiation, microwave radiation, infrared radiation, visible light information, ultraviolet radiation, x-ray radiation, alpha radiation, beta radiation, gamma radiation, and fluorescence radiation.

15. The method of claim 12 wherein positioning the coiled configuration portion comprises sliding the guidewire longitudinally with respect to the blood vessel.

16. The method of claim 12 wherein positioning the coiled configuration portion comprises rotating the guidewire about a central axis.

17. The method of claim 12 wherein the coiled configuration portion comprises a corkscrew configuration wherein the guidewire is coiled a plurality of turns around a central axis.

18. The method of claim 12 wherein the coiled configuration portion is adapted to expand radially toward a blood vessel wall.

19. The method of claim 12 wherein determining presence of vulnerable plaque comprises comparing received information from a healthy blood vessel site to received information from a vulnerable plaque site.

20. The method of claim 12 further comprising transmitting the received information from a diagnostic site to a site external the patient.

21. The method of claim 12 further comprising delivering a therapeutic agent to the blood vessel based on the information.

22. A system for detecting a vulnerable plaque associated with a blood vessel of a patient, the system comprising:
a catheter; and
a flexible guidewire slidably carried by the catheter, the guidewire including a coiled configuration means for positioning at least one sensor;
wherein the coiled configuration means are positioned adjacent a wall of the blood vessel to allow the sensor to receive information for determining presence of the vulnerable plaque.
